(19) United States
(12) Patent Application Publication
(10) Pub. No.: US 2025/0037807 A1
(43) Pub. Date: Jan. 30, 2025

(54) CHEMICAL SEARCH AND PROPERTY PREDICTION

(71) Applicant: TensorSpace, Inc., Fairfax, VA (US)

(72) Inventor: Jonathan Judah Ben-Joseph, Fairfax, VA (US)

(21) Appl. No.: 18/359,570

(22) Filed: Jul. 26, 2023

(51) Int. Cl. G16C 20/90; G16C 20/70; G16C 20/80

(52) U.S. Cl. CPC G16C 20/90; G16C 20/70; G16C 20/80

(57) ABSTRACT

Machine learning can be used to identify and/or predict the properties of molecules. A mathematical model is trained to generate a combined embedding space that includes language embeddings and molecule representation embeddings. The mathematical model may be a fine-tuned language model. The fine-tuned language model may receive queries related to molecules and properties of molecules and provide predictions of properties of the queries molecules, similar molecules, and/or may find molecules with a desired property.

### BACKGROUND

Many chemical properties cannot be calculated analytically and are currently imperfectly determined through in-vitro assays or animal models. In-vitro assays and animal models can take months to complete, can be extremely costly, and raise ethical concerns. Accordingly, it may be desirable to develop alternative methods for predicting the chemical properties of molecules.

### SUMMARY

In some aspects, the techniques described herein relate to a computer-implemented method of querying for molecules, including: obtaining a mathematical model for embedding text and molecule representations in an embedding space; receiving a natural language search query, wherein the search query includes a text description of a chemical property of a molecule; computing, using the mathematical model, an embedding of the search query; identifying, using a distance metric, a candidate embedding in the embedding space that are in proximity to the embedding of the search query; obtaining a candidate molecule corresponding the candidate embedding; and generating, for presentation at a user interface, a representation of the candidate molecule in response to the search query.

In some aspects, the techniques described herein relate to a method, wherein computing the embedding of the search query includes processing the text description with a sentence embedding model.

In some aspects, the techniques described herein relate to a method, wherein the embedding is a transformer text embedding.

In some aspects, the techniques described herein relate to a method, wherein the candidate embedding is representative of a SELFIES or a SMILES string of a molecule.

In some aspects, the techniques described herein relate to a method, further including: identifying, using the distance metric, a second candidate embedding in the embedding space that is in proximity to the embedding of the candidate molecule; obtaining a property description corresponding to the second candidate embedding; and generating, for presentation at the user interface, a depiction of the property description.

In some aspects, the techniques described herein relate to a method, wherein the distance metric includes cosine similarity.

In some aspects, the techniques described herein relate to a method, wherein the representation of the candidate molecule includes a graphical depiction of a structure of the molecule.

In some aspects, the techniques described herein relate to a method, wherein the mathematical model includes a transformer-based model.

In some aspects, the techniques described herein relate to a method, wherein the mathematical model includes a model trained using negative descriptions of molecules.

In some aspects, the techniques described herein relate to a method, wherein the mathematical model includes a model trained using a pseudo-normal negative loss function.

In some aspects, the techniques described herein relate to a computer-implemented method of querying for properties of a molecule, the method including: receiving a molecule search query, wherein the molecule search query includes a text string representative of the molecule; tokenizing the text string; computing, using a mathematical model, an embedding of the tokenized string; identifying, using a distance metric, a candidate embedding in an embedding space that is in proximity to the embedding of the tokenized string; mapping the candidate embedding to a text of a candidate molecule property; and generating, for presentation at a user interface, a representation of the candidate molecule property.

In some aspects, the techniques described herein relate to a method, wherein computing the embedding of the search query includes processing the text string representation with a sentence embedding model.

In some aspects, the techniques described herein relate to a method, wherein the embedding is a transformer text embedding.

In some aspects, the techniques described herein relate to a method, wherein the candidate embedding is representative of a SELFIES or a SMILES string of a molecule.

In some aspects, the techniques described herein relate to a method, wherein the distance metric includes cosine similarity.

In some aspects, the techniques described herein relate to a method, wherein the mathematical model includes a model trained using negative descriptions of molecules.

In some aspects, the techniques described herein relate to a system, including: at least one server computer including at least one processor and at least one memory, the at least one server computer configured to: obtain a mathematical model for embedding text and molecule representations in an embedding space; receive a natural language search query, wherein the search query includes a text description of a chemical property of a molecule; compute, using the mathematical model, an embedding of the search query; identify, using a distance metric, a candidate embedding in the embedding space that is in proximity to the embedding of the search query; obtain a candidate molecule corresponding to the candidate embedding; and generate, for presentation at a user interface, a representation of the candidate molecule in response to the search query.

In some aspects, the techniques described herein relate to a system, wherein the at least one server computer is configured to compute the embedding of the text by processing the text with a sentence embedding model.

In some aspects, the techniques described herein relate to a system, wherein the embedding space is a transformer embedding space.

In some aspects, the techniques described herein relate to a system, wherein the candidate embedding is representative of a SELFIES or a SMILES string of a molecule.

In some aspects, the techniques described herein relate to a computer-implemented method for training an embedding model, including: obtaining a data set including at least a first data element, wherein the first data element includes a first chemical structure representation of a first molecule and a first natural language description of at least one property of the first molecule; generating a first negative natural language description of the first molecule; generating a first positive training sample including the first chemical structure representation of the first molecule and the first natural language description; generating a first negative training sample including the first chemical structure representation of the first molecule and the first negative natural language description; and training the embedding model, wherein the training includes: processing the first chemical structure representation of the first molecule using the embedding model to generate a first molecule embedding; processing the first natural language description using the embedding model to generate a first text embedding; processing the first negative natural language description using the embedding model to generate a second text embedding; computing a first error value from the first molecule embedding and the first text embedding; computing a second error value from the first molecule embedding and the second text embedding; and updating parameters of the embedding model using the first error value and the second error value.

In some aspects, the techniques described herein relate to a method, further including computing the first error value and the second error value using triplet loss.

In some aspects, the techniques described herein relate to a method, further including computing the first error value and the second error value using a pseudo-normal negative loss function.

In some aspects, the techniques described herein relate to a method, wherein the pseudo-normal negative loss function assigns values for the first negative natural language description from a part of a normal curve.

In some aspects, the techniques described herein relate to a method, further including tokenizing the first chemical structure representation into a sequence of tokens.

In some aspects, the techniques described herein relate to a method, wherein the first chemical structure representation includes a SMILES string.

In some aspects, the techniques described herein relate to a method, wherein the first chemical structure representation includes a SELFIES string.

In some aspects, the techniques described herein relate to a method, further including: generating a second negative natural language description of the first molecule; generating a second negative training sample including the first chemical structure representation of the first molecule and the second negative natural language description; and training the embedding model, wherein the training includes: processing the second negative natural language description using the embedding model to generate a third text embedding; computing a third error value from the first molecule embedding and the third text embedding; and updating the parameters of the embedding model using the third error value.

In some aspects, the techniques described herein relate to a method, further including: obtaining a second data element from the data set, wherein the second data element includes a second chemical structure representation of a second molecule and a second natural language description of at least one property of the second molecule; generating a second negative natural language description of the second molecule; generating a second positive training sample including the second chemical structure representation of the first molecule and the first natural language description; generating a first negative training sample including the second chemical structure representation of the first molecule and the second negative natural language description; and training the embedding model, wherein the training includes: processing the second chemical structure representation of the first molecule using the embedding model to generate a second molecule embedding; processing the second natural language description using the embedding model to generate a third text embedding; processing the second negative natural language description using the embedding model to generate a fourth text embedding; computing a third error value from the second molecule embedding and the third text embedding; computing a fourth error value from the second molecule embedding and the fourth text embedding; and updating parameters of the embedding model using the third error value and the fourth error value.

In some aspects, the techniques described herein relate to a method, wherein the first natural language description includes at least one of a toxicity, an HIV effect, an Alzheimer's effect, a side effect, a clinical trial outcome, or a blood-brain barrier permeability of the first molecule.

In some aspects, the techniques described herein relate to a method, wherein obtaining the data set includes scraping the first chemical structure representation from public data sources and using surrounding text of the first molecule as the first natural language description.

In some aspects, the techniques described herein relate to a system, including: at least one server computer including at least one processor and at least one memory, the at least one server computer configured to: obtain a data set including at least a first data element, wherein the first data element includes a first chemical structure representation of a first molecule and a first natural language description of at least one property of the first molecule; generate a first negative natural language description of the first molecule; generate a first positive training sample including the first chemical structure representation of the first molecule and the first natural language description; generate a first negative training sample including the first chemical structure representation of the first molecule and the first negative natural language description; and train an embedding model, wherein the training includes: processing the first chemical structure representation of the first molecule using the embedding model to generate a first molecule embedding; processing the first natural language description using the embedding model to generate a first text embedding; processing the first negative natural language description using the embedding model to generate a second text embedding; computing a first error value from the first molecule embedding and the first text embedding; computing a second error value from the second molecule embedding and the second text embedding; and updating parameters of the embedding model using the first error value and the second error value.

In some aspects, the techniques described herein relate to a system, wherein the at least one server computer is configured to compute the first error value and the second error value using triplet loss.

In some aspects, the techniques described herein relate to a system, wherein the at least one server computer is configured to compute the first error value and the second error value using a pseudo-normal negative loss function.

In some aspects, the techniques described herein relate to a system, wherein the pseudo-normal negative loss function assigns values for the first negative natural language description from a part of a normal curve.

In some aspects, the techniques described herein relate to a system, wherein the at least one server computer is configured to tokenize the first chemical structure representation into a sequence of tokens.

In some aspects, the techniques described herein relate to a system, wherein the first chemical structure representation includes a SMILES string.

In some aspects, the techniques described herein relate to a system, wherein the first chemical structure representation includes a SELFIES string.

In some aspects, the techniques described herein relate to a system, wherein the at least one server computer is further configured to: generate a second negative natural language description of the first molecule; generate a second negative training sample including the first chemical structure representation of the first molecule and the second negative natural language description; and train the embedding model, wherein the training includes: processing the second negative natural language description using the embedding model to generate a third text embedding; computing a third error value from the first molecule embedding and the third text embedding; and updating the parameters of the embedding model using the third error value.

In some aspects, the techniques described herein relate to a system, wherein the first natural language description includes at least one of a toxicity, an HIV effect, an Alzheimer's effect, a side effect, a clinical trial outcome, or a blood-brain barrier permeability of the first molecule.

### BRIEF DESCRIPTION OF THE FIGURES

The invention and the following detailed description of certain embodiments thereof may be understood by reference to the following figures:

FIG. 1A is an example system for identifying molecules properties using the techniques described herein.

FIG. 1B is a flowchart for data execution for an example molecule search system.

FIG. 2 is an example of an interface for submitting queries and receiving query results.

FIG. 3 shows aspects of the embedding space of the trained language models.

FIG. 4 is an example system for training the model.

FIG. 5A is a flow diagram depicting aspects of generating positive and negative training samples.

FIG. 5B is a flow diagram depicting aspects of generating a triplet sample.

FIG. 6 is a flow diagram depicting aspects of training with a pseudo-normal negative loss function.

FIG. 7 is a flowchart of an example method of querying for molecules using a trained mathematical model.

FIG. 8 is a flowchart of an example method of training an embedding model.

FIG. 9 is a flowchart showing details of an example training method.

### DETAILED DESCRIPTION

Chemical property analysis is used in various applications and fields such as therapeutic drug development, material sciences, photovoltaics development, battery science, manufacturing, food science, and many others. In various scenarios, a person or user may need to identify the properties of molecules or identify molecules with specific properties.

In one example, therapeutic drug development requires the identification of molecule toxicity. Chemically active molecules are often desirable for therapeutics, but highly reactive compounds may be toxic. Therapeutic drug development requires a search for chemically active molecules that do not interfere with the body's normal functioning.

However, some chemical properties cannot be calculated analytically. In the case of drug development, molecule properties are determined through expensive in-vitro assays or animal models, which are not only costly but also raise ethical concerns. In other fields, molecule property research requires extensive and time-intensive experimentation and testing. These challenges have prompted the development of the alternative methods and systems described herein that facilitate a faster and less costly identification of the chemical properties of a molecule.

The techniques described herein use machine learning to identify and/or predict the properties of molecules. In one example, the techniques described herein can be used in screening molecules for drug development, helping to narrow down potential therapeutics by eliminating likely toxic candidates and reducing the need for costly in-vitro (lab) experiments. In another example, the techniques described herein can be used to optimize specific properties (such as blood-brain barrier penetration for brain-targeting drugs) and can also help select molecules that appear to interact or disrupt a particular protein target.

The techniques described herein can accelerate and reduce the cost associated with the screening of molecules and the identification of properties of molecules. Accelerating the screening of new molecules, especially during early development stages, can significantly enhance the development of new drugs, materials, and devices.

The techniques described herein can be applied to any type of chemical compound in any field. The examples described herein may include determining the properties of small molecules for drug development, but the techniques described herein are not limited to these examples. The techniques described herein can be applied to various types of molecules in the fields of material science, drug development, food science, device development, and the like.

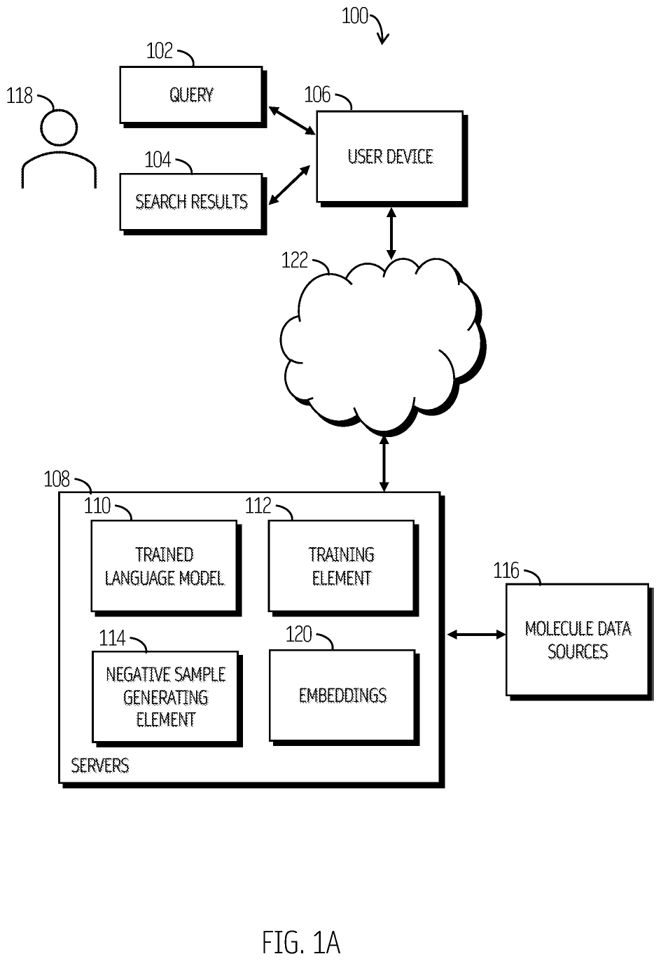

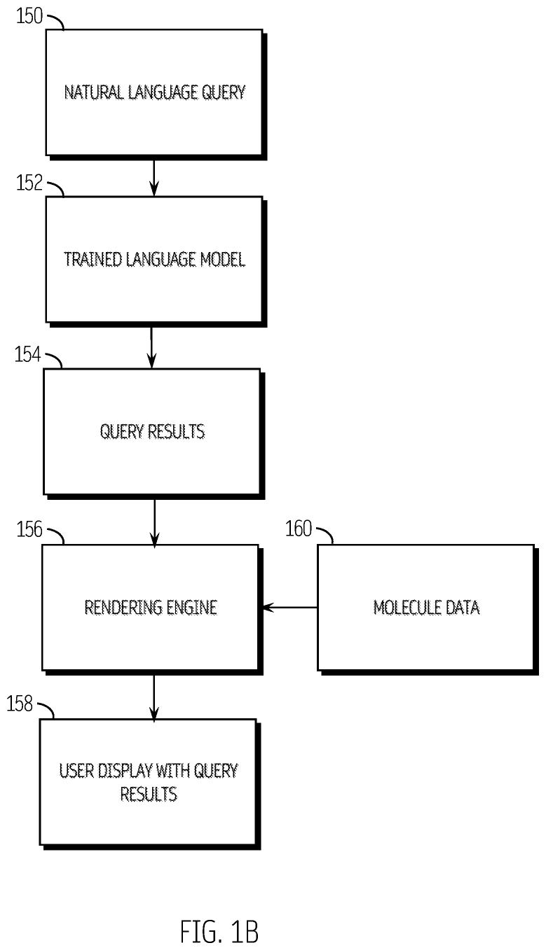

FIG. 1A is an example system 100 for identifying molecule properties using the techniques described herein. In FIG. 1A, user 118 performs a molecule search from a user device 106. User device 106 may be any appropriate computing device, such as a computer, tablet, or phone. User device 106 may be running any appropriate software to allow user 118 to submit a query 102 via user device 106. The user device 106 may submit the query 102 to a mathematical model such as a trained language model 110. The trained language model 110 may return a search result 104 to the user device 106 in response to the query 102.

In one example, query 102 may be a natural language query and may include words, numbers, phrases, and/or chemical representations of molecules. The query 102 may be a natural language search string requesting a search for molecules or information about one or more molecules. The search string may be processed by the trained language model 110 to identify molecules that match the query string and return a search result 104. The search result 104 may identify one or more molecules.

In another example, the query 102 may include molecule data that may include the chemical composition of a molecule and/or the structure of the molecule. In one example, a query may include a representation of a molecule. The representation of the molecule of query 102 may be processed by the trained language model 110 to identify properties of the molecule and/or other molecules with similar properties to that of the molecule in the query 102. The search result 104 may identify one or more properties of the molecule and/or other molecules with similar properties to that of the molecule in the query 102.

In some implementations, user device 106 may process the query 102 using installed software and trained language model in a standalone manner such that no other devices are assisting in the processing of query 102. In some implementations, user device 106 may use a service provided over a network connection. For example, user device 106 may use network 122 to access one or more servers 108 that can execute a trained language model 110 to process the query 102 and return the results to the user device 106.

The trained language model 110 may be trained using the computing resources of one or more servers 108. Training may be facilitated by a training element 112, embeddings storage 120, and a negative sample generating element 114.

FIG. 1B is a flowchart for data execution for an example molecule search system. As a first step, a natural language search query is generated 150 by a user. The natural language search query may include different types of queries composed for different purposes and may have varying complexity, structure, and data elements. A natural language search query may be a text string.

In one example, a query may be structured with the purpose of identifying molecules with one or more desired properties. The search query may include a description of one or more properties of a molecule. The query may be a text string that includes a list of properties, a sentence description of a property, a question that includes at least one property, and/or the like. In one example, the sentence description may relate to at least one of toxicity, an HIV effect, an Alzheimer's effect, a side effect, a clinical trial outcome, or a blood-brain barrier permeability of the first molecule. The search query may include negative descriptions of properties describing properties that are not desirable or should be excluded. The search query may include a description of any property of the molecule, such as toxicity, permeability, solubility, bioavailability, stability, protein binding, and the like. In one example, a query may be a string such as “HIV therapeutic that is brain barrier permeable and low toxicity.”

In another example, a query may be structured with the purpose of identifying the properties of a molecule. The search query may be a molecule search query and may include a text string that is representative of a molecule. Representations of molecules may include text string representations such as SMILES (Simplified Molecular-Input Line-Entry System) strings, SELFIES (Self-referencing embedding strings), or any other suitable molecule representation. The representations of the molecules may be in human-readable format. The search query may include natural language along with molecule representations. The natural language may constrain the search for properties of the molecule in the query. For example, the query may include a SMILES string for a molecule and ask a question about a property of a molecule: “Is CC(═O) OC1=CC═CC═C1C(═O)O toxic?”

In another example, a query may be structured with the purpose of comparing the properties of two or more molecules. The search query may include two or more representations of a molecule and may include text strings that are representative of molecules. The search query may include one or more representations of a molecule and the name of a molecule. The search query may include any number of descriptions, names, and natural language that may limit the comparison to a set or properties of categories of properties.

After a query is generated, the query may be processed by a mathematical model, such as a trained language model 152. In some implementations, the query may be preprocessed before inputted to a trained language model. Preprocessing may include error checking of the query (i.e., spelling corrections, verification of SMILES syntax), tokenization of the query, reformatting, and the like. In some implementations, queries may be processed by the trained language model immediately or in real-time after they are received. In some implementations, processing with the trained language model may be executed in a batch mode where the processing is executed when a number of queries of received or according to a batch processing schedule.

The trained language model may be a natural language model that is trained on the joint distribution of natural language and chemical representation of molecules. The chemical representations of molecules may be language-like representations such as SMILES strings. The trained language model may be trained on one or more types of chemical representations of molecules. In some implementations, the types of chemical representations entered into a query may have to correspond to the types of chemical representations on which the model was trained. In some implementations, a chemical representation translator may be used during preprocessing of a query to translate a chemical representation of a query into a chemical representation that was used during the training of the language model.

Processing of the query with the language model may generate an output with the query results 154. The query results 154 may be based on the embeddings generated by the language model during the processing of the query. Query results may be identified using a distance metric in an embedding space and the query results may be determined from matching embeddings that are in a defined proximity to the embeddings computed by the language model. Matching embeddings of the language model may correspond to one or more words, phrases, sentences, properties, and/or molecule descriptions. The query results may correspond to the embeddings that are closest or in a defined proximity to the embeddings generated from the query. In some implementations, embeddings that are in a defined proximity (i.e., the top 10 closest embeddings) to the embeddings generated from the query may be scored using a scoring function. The query results may correspond to the highest scoring or most probable embeddings that are in proximity to the embedding computed for the search query. Scoring of the scoring function may be related to the type of element of each embedding (i.e., property of molecule versus molecule representation).

In one example, the query results for a query that was structured with the purpose of identifying molecules with one or more desired properties may include the chemical representations of molecules that match and/or are predicted to match the desired properties. In another example, the query results for a query that was structured with the purpose of identifying the properties of a molecule may include properties and/or descriptions of properties that match and/or are predicted to match the queried chemical description. In another example, the query results for a query that was structured with the purpose of comparing the properties of two or more molecules may include properties and/or descriptions that contrast the properties and/or predicted properties of the molecules.

The query results 154 may be further processed by a rendering engine 156. The rendering engine 156 may format, post-process, enhance, and/or enrich the query results. Results may be enhanced with data from other databases or other sources such as molecule data 160 sources to include data associated with molecule data in the query results. The rendering engine 156 may be configured to generate graphical interfaces or depictions for reviewing and/or manipulating the query results. Outputs of the rendering that include the query results may be displayed to the user 158. In some implementations, the query results may be directly provided to a user and/or another process for analysis or use with downstream tools.

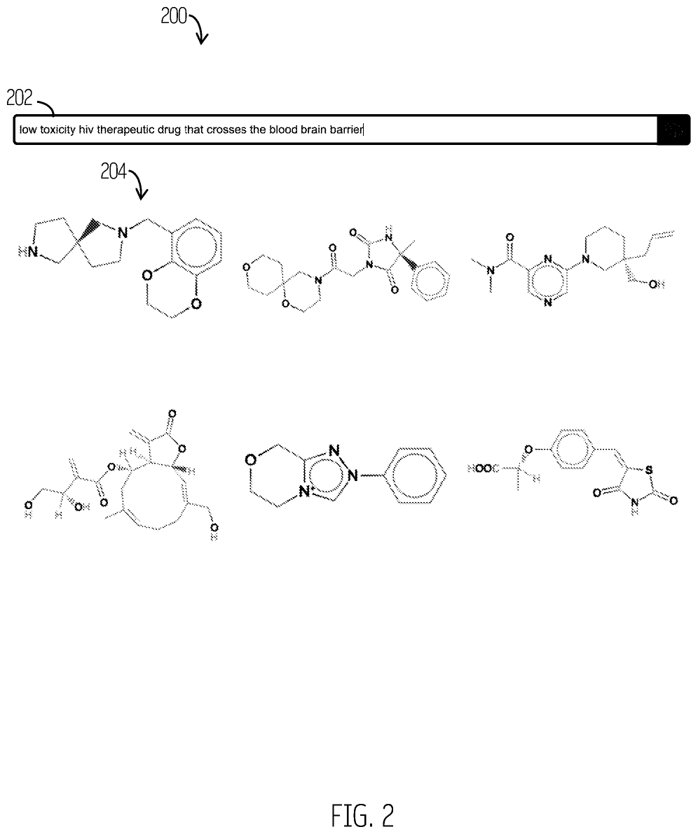

FIG. 2 is an example of an interface 200 that may be used by a user to submit queries and receive query results. The Interface 200 may include one or more query entry elements. In one example, a query entry interface may include query entry box 202 for entering natural language and/or chemical representation of molecules. In some implementations, any number of entry elements may be used and may include textboxes, lists, drop-down boxes, and the like. The interface may further include one or more results elements. In one example, results elements may include a results list 204. The results list 204 may include a list or a gallery of results and may include graphical depictions of the molecules and/or their properties. The elements of the results list 204 may be selectable and may show additional information about molecules when selected. The interface 200 may be implemented using any suitable technology and may be part of an application, executable in a browser, a plug-in, and the like.

The trained language models described herein are trained to generate and operate in a combined embedding space where language and chemical representations of molecules are mapped to a common vector space. An embedding of an element is a vector in a vector space that represents an element (such as a word, phrase, chemical representation, etc.) but does so in a manner that preserves useful information about the elements and their relationships. The trained language models described herein operate on a combined embedding space where embeddings are constructed so that words relating to properties of molecules and chemical representations of molecules are close to one another in the vector space. The embedding space may further be constructed so that chemical representations of molecules with similar properties are close to one another in the vector space.

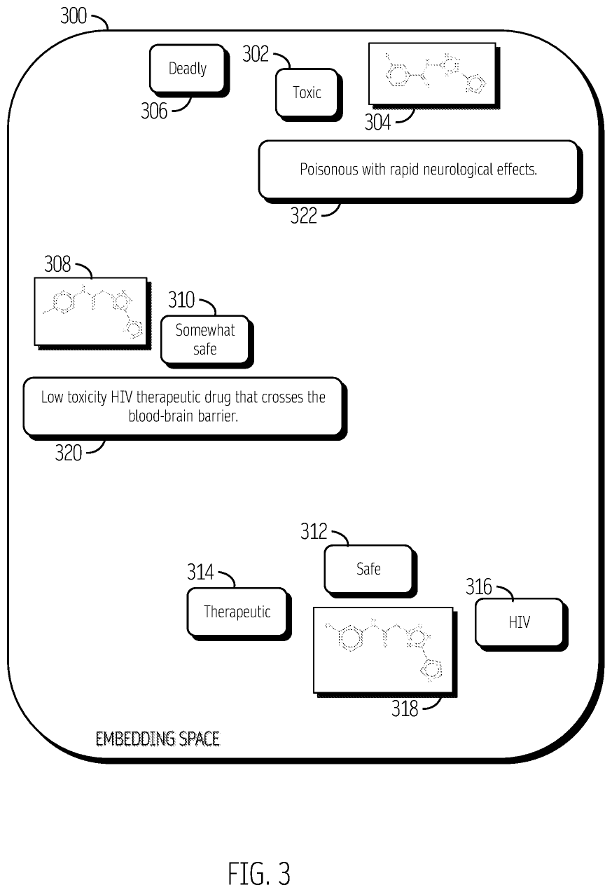

FIG. 3 shows aspects of the embedding space of the trained language models used herein. The embedding space 300 is a high-dimensional vector space in which elements are represented as points, or “embeddings.” In the example of FIG. 3, for clarity of presentation, the embedding space is shown as a two-dimensional vector space, but an embedding space may be of any dimension and may use larger vector spaces, such as a 128-dimensional vector space or a 512-dimensional vector space. The embeddings are vectors that capture some of the essential qualities or features of the elements they represent, and their arrangement in the embedding space reflects the relationships between these elements. The embedding space 300 includes embeddings of language elements 302, 306, 322, 310, 320, 312, 314, 316 and representations of molecules 304, 308, 318. Embeddings of language elements may be embeddings of sentences, words, phrases, and the like that are representative of the properties of molecules. Embeddings of representations of molecules may be embeddings of SMILES strings, for example. Vectors of elements that are similar may be relatively close to one another compared to elements that are different from one another. For example, molecule 318 and language elements 312, 314, and 316 relate to the same therapeutic and are close to each other in the embedding space 300, while different elements, such as molecule 304 and language elements 302, 306, and 322 relate to a toxic poison and are at a far distance in the space 300.

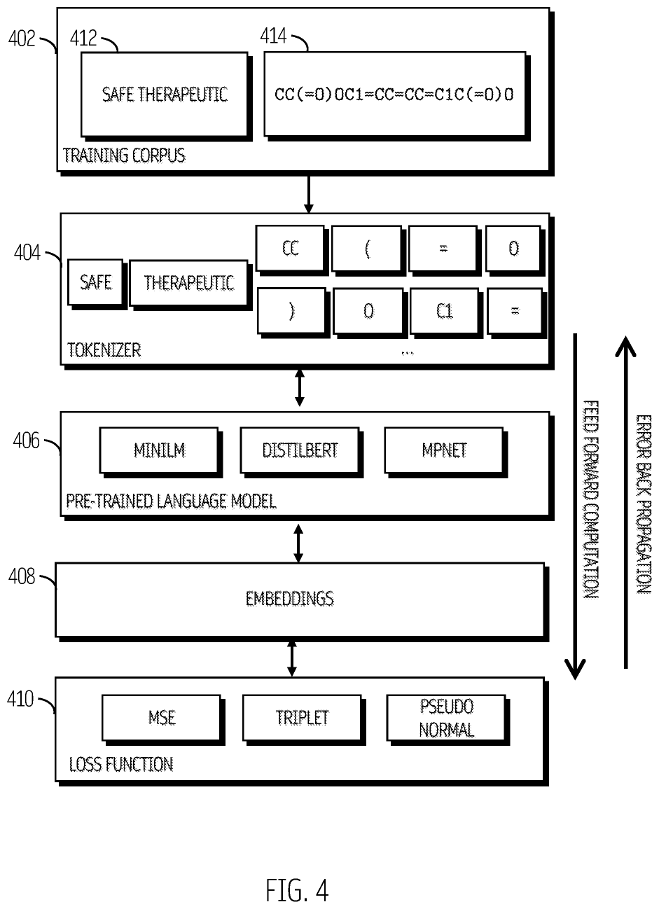

The embeddings are learned during the training process of a model. FIG. 4 is an example system 400 for training the model. Training may involve fine-tuning of a pre-trained language model 406. A pre-trained language model is a model that has been previously trained on a large corpus of text data and is trained to learn the statistical properties of the language, such as word co-occurrence, sentence structures, and other language patterns. During pre-training, a language model is typically trained on a task like predicting the next word in a sentence given the previous words. Pre-training may be unsupervised. Any suitable pre-trained language model may be used and may include sentence embedding models and may include transformer-based models. In one example, a pre-trained model may be a transformer-based model that generates transformer text embeddings in a transformer embedding space. Examples of suitable pre-trained language models include models such as MPNet, DistilBERT, and miniLM.

A pre-trained model is fine-tuned with a training corpus 402 that includes chemical representations of molecules 414 and language 412 that identifies aspects such as the properties of the represented molecule 414. During fine-tuning using the training corpus 402, the pre-trained language model learns to create embedding vectors for the chemical representations of the molecules and adjusts the embeddings of the language embeddings. Fine-tuning involves supervised training on a smaller training corpus 402 where the pre-trained weights of the pre-trained language model 406 are adjusted. One advantage of using pre-trained language models for training is that they can leverage the knowledge of the language they learned during pre-training, and the trained model can achieve a high level of performance even when the size of the training corpus 402 is small.

The training corpus 402 may include data from molecule databases such as MoleculeNet and other sources. In some implementations, the training corpus 402 may be derived from molecule database data. In one example, data may be derived by mapping ratings or numerical values associated with molecules to words or natural language descriptions.

During training, the data samples of the training corpus 402 are tokenized using one or more tokenizers 404. The tokenizer 404 breaks down the elements of each sample from the training corpus 402 into a sequence of smaller units called tokens. Tokens of the language 412 are often words, but they can also be phrases, sentences, parts of words, or individual characters. Tokens of the chemical representations of molecules may be of various granularity. In some implementations, the chemical representations of molecules may be SMILES strings, and tokens may correspond to each atom and bond description of the string. In one example, data samples may be a text string and the tokenizer may generate a tokenized string that break down the text string into a sequence of tokens.

Tokenized training samples may be provided as inputs to the pre-trained language model 406. The pre-trained language model 406 generates embeddings 408 from the inputs. The embeddings 408 are further provided to a loss function 410. The loss function 410 is configured to measure the discrepancy between the model's current representation of the data (the embeddings) and the desired representation. Parameters of the models are adjusted to minimize the loss function using stochastic gradient descent (SGD) and backpropagation or variants thereof. SGD may be used to adjust the model's parameters in the direction that reduces the loss, a direction indicated by the gradient of the loss function with respect to the parameters. This process is repeated iteratively and may include one or more epochs until the training converges (i.e., the loss stops decreasing or reaches an acceptable level). In implementations, various loss functions may be used, such as a triplet loss function, mean squared error (MSE) loss function, pseudo normal loss function, and the like.

In some implementations, generating a training corpus 402 may include generating positive and negative training samples. In some implementations, negative training samples may improve the convergence of the model parameters. In some implementations, generating negative training samples may reduce the number of training samples required for the training of a model to converge. In some implementations, there may not be enough positive data samples for the training to converge; however, negative training samples may be synthesized from training data and the negative training samples may reduce the training data requirements for the training to converge.

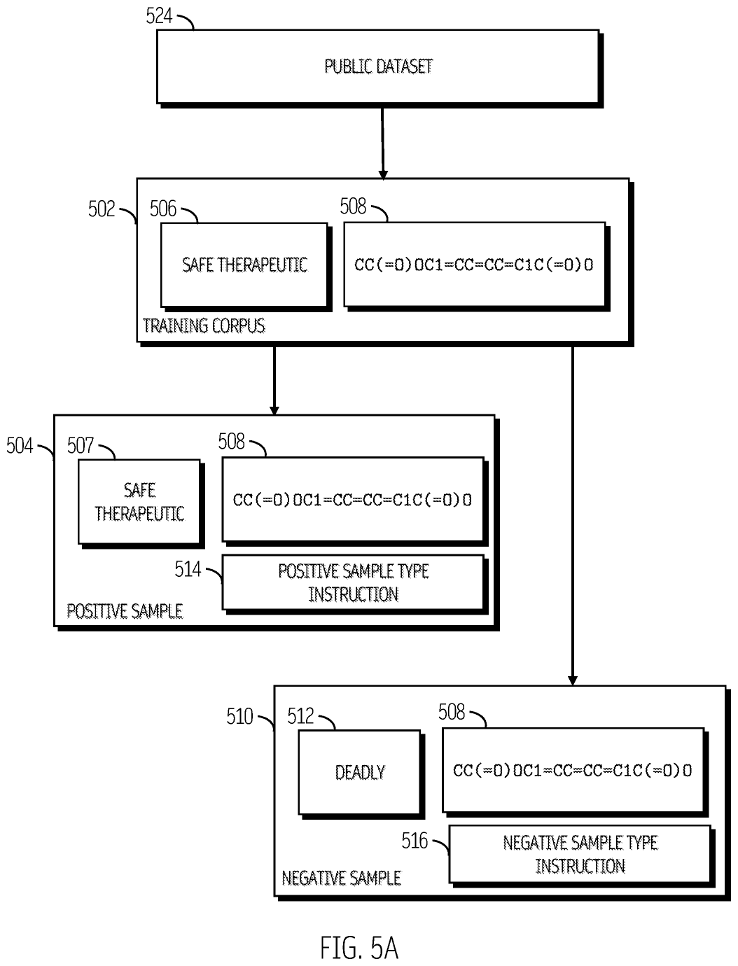

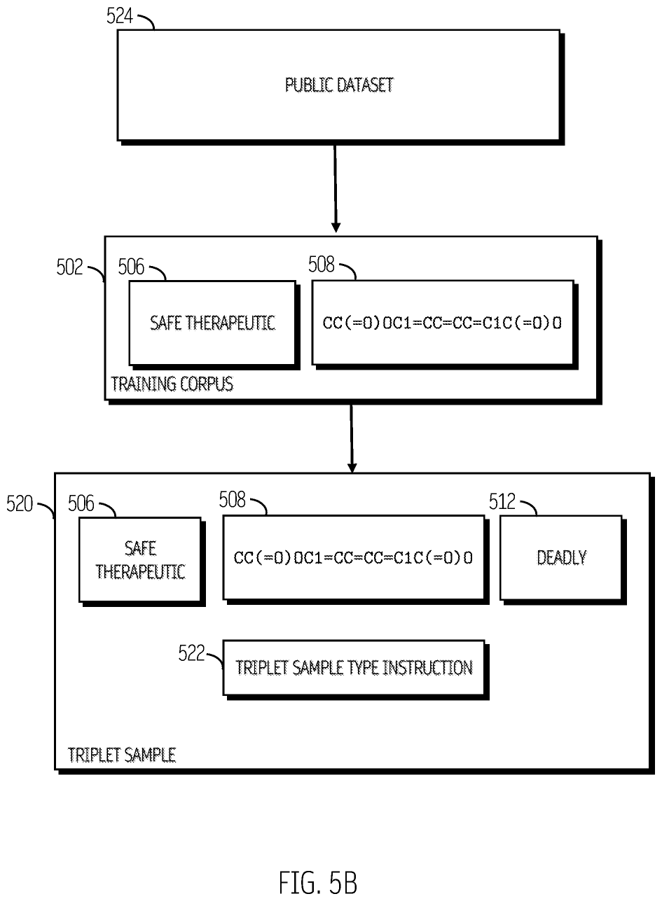

FIG. 5A is a flow diagram depicting aspects of generating positive and negative training samples. A training corpus 502 may be derived from public datasets 524. A training corpus may include positive and negative samples. Positive samples are examples that belong to the class or condition of interest. Positive samples may be pairs of examples of molecules properties and chemical representations of molecules that the model needs to identify correctly. Negative samples are examples that do not belong to the class of interest. Negative samples may be pairs of molecules properties and chemical representations of molecules that are not accurate or where the molecule property description does not accurately describe the molecule represented by the chemical representation. Positive and negative samples may be directly derived from a public dataset. A public data set may include language that describes a property of a molecule and language that describes which property the molecule is lacking. The respective language may be selected to directly generate positive and negative samples.

Negative samples may also be synthesized from positive examples of a training corpus 502. A training corpus may include a positive example of a language description 506 (i.e. a property of a molecule) that accurately describes an aspect of the molecule of the representation 508. The positive example of a training corpus 502 may be used as a positive training sample 504 by including positive language description 507, and the chemical representation 508 of the molecule. The positive example of a training corpus 502 may be used to synthesize a negative training sample 510. The negative training sample 510 may include the chemical representation 508 of the molecule and a language description 512 that does not accurately describe the molecule of the chemical representation 508. The language description 512 of the negative sample may be synthesized from the language description 507 of the positive sample 504. The language description 512 may be selected such that it describes an opposite property to that of the language description 506 found in the positive example of the training corpus 502.

The language description 512 for the negative sample 510 may be generated by processing the language description 507 of the positive example 505 with a language model trained to generate opposing descriptions. The language description 512 for the negative sample 510 may be generated by randomly generating a language description 506 or randomly selecting a language description from a list of possible language descriptions (and possibly adding checks to ensure that the randomly generated language description is not a correct description of the molecule).

The positive sample 504 and the negative sample 510 may be associated with a label that identifies the samples as positive or negative, respectively. In one example, the positive sample 504 may be associated with a sample type instruction 514 that identifies the sample as a positive sample, and the negative sample 510 may be associated with a sample type instruction 516 that identifies the sample as a negative sample. For positive samples, the model should ideally predict a low loss value, and for negative samples, it should predict a high loss value. The loss function penalizes deviations from these ideal predictions. The sample type instructions may be used to update model parameters, depending on the model output for each sample during training.

Training samples may be derived from various public molecular datasets. In some cases, training samples may be derived by scraping chemical structure representations of molecules from public data sources (i.e., publications, databases) and using surrounding text as a description for the molecules.

FIG. 5B is a flow diagram depicting aspects of generating a triplet sample. A triplet sample 520 may be generated from a positive example from a training corpus 502. A triplet sample 520 may be generated by synthesizing a negative language description 512 from the language description 506 of the positive example of the training corpus 502. A negative language description 512 may be generated using any appropriate techniques, such as those described with respect to descriptions of FIG. 5A. A triplet sample may be generated by combining the positive language description 506 of the positive example of the training corpus 502, the chemical representation 508 of the molecule of the positive example of the training corpus 502, and the negative language description 512 that may be synthesized from the positive language description 506. The triplet sample 520 may further include a triplet sample type instruction 522, which may identify the positive and negative descriptions of the triplet. During training with a triplet sample, a triplet loss function may be used. The goal of the triplet loss function may be to make the distance (i.e., Euclidean or cosine distance) between the chemical representation of the molecule and the positive language description in the embedding space smaller than the distance between the chemical representation of the molecule and the negative language description.

In some implementations, training with negative samples may include the use of a pseudo-normal negative loss function. A pseudo-normal negative loss function, as described herein, can improve the performance of the embedding model compared to other loss functions, such as the triplet loss function. The loss function is referred to as a pseudo-normal loss function because it is based on a modified normal distribution. The pseudo-normal negative loss function may be configured to return a value close to 1 for positive samples. The pseudo-normal negative loss function may be configured to further return values selected from the normal distribution for the negative samples.

The pseudo-normal negative loss function may be based on a normal distribution N (μ=0, σ=2) that has the following probability density function:

$$N ( μ = 0 , σ = 2 ) = \frac{1}{σ \sqrt{2 π}} {e}^{- \frac{1}{2} {( \frac{x - μ}{σ} )}^{2}} = \frac{{e}^{- {x}^{2} / 8}}{2 \sqrt{2 π}} ,$$

where μ is the mean and σ is the standard deviation.

The loss function uses a scale factor γ based on the normal distribution above. In one example, the maximum of the scale factor may be set as 0.999 as the true positive value and the scaled top of the normal curve. In some training examples, a scale factor that is very close to 1 but not exactly 1 may improve the speed at which the training converges. In one example, the scale factor γ may be defined as:

$$γ = 0.999 \frac{1}{N ( x = 0 , μ = 0 , σ = 2 )} .$$

The pseudo-normal negative loss function for a label ŷ may be defined as:

$$\hat{y} = N ( x = \vert y - i \vert , μ = 0 , σ = 2 ) * γ$$

where y is the ground truth and i is the index of the prompt. For positive examples, the pseudo-normal negative loss function will return a value of 0.999, but negative samples with return lower numbers as part of the normal curve.

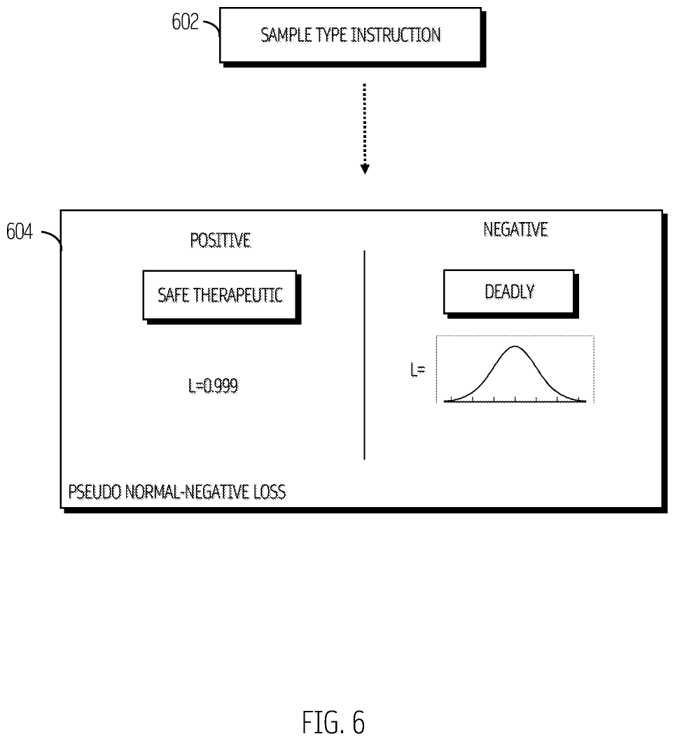

FIG. 6 is a flow diagram depicting aspects of training with a pseudo-normal negative loss function. The sample type instruction 602 for a pseudo-normal negative loss function may be different for positive samples and negative samples during training. For positive samples (left side of 604), the function may return a value close to 1, such as a value of 0.999. For negative samples (right side of 604), the function may return a value that is less than the value returned for positive samples and may be selected based on a normal distribution. The selection of values selected from the normal distribution for the negative sample may be determined based on the negative language description of the negative sample.

In one example, the selection of values selected from the normal distribution for the negative sample may be determined using a function that measures how far away the negative language description is from the positive language description. In some cases, the language descriptions may be associated with an index of a list of language descriptions, and the function may be based on a difference between the indexes of the positive language description and the negative language description.

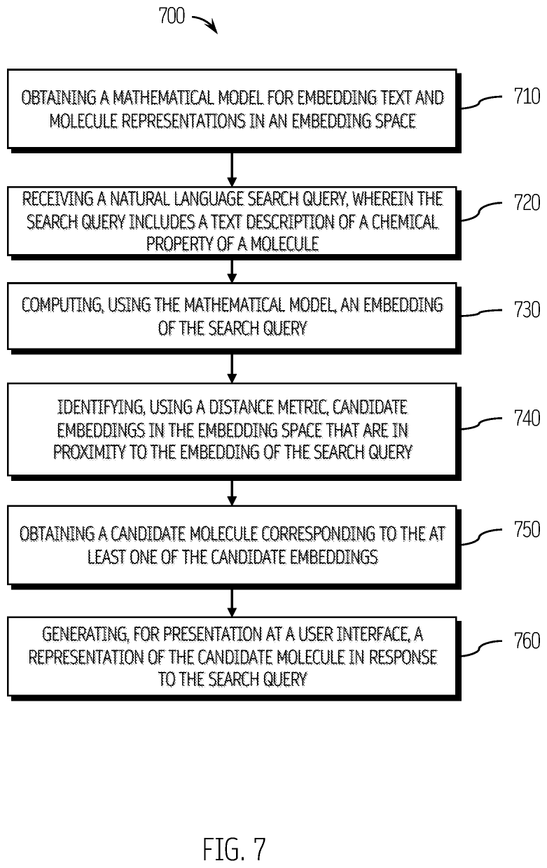

FIG. 7 is a flowchart of an example method 700 of querying for molecules using a trained mathematical model. In one example, method 700 may be implemented by the systems and models described with respect to FIGS. 1-3. At step 710, a mathematical model for embedding text and molecule representations in an embedding space is obtained. As described herein, the mathematical model may be a fine-tuned language model and may be a transformer-based model that generates sentence embeddings and/or transformer text embeddings. The mathematical model may be a model trained as described with respect to FIG. 4 herein. The mathematical model may be a model that is trained to generate embeddings in a combined embeddings space that includes embeddings of language and embeddings of molecule representations or chemical representations.

At step 720, a natural language search query may be received. The natural language search query may be structured with the purpose of identifying molecules with one or more properties and may include a text description of a chemical property of a molecule. The natural language search query may be structured with the purpose of identifying the properties of molecules and may include representations of molecules such as SMILES string. The natural language search query may be structured with the purpose of comparing two or more molecules and may include two or more representations of molecules such as SMILES string. In embodiments, the natural search query may include any combination of text descriptions and molecule representations, The search query may be received from a user interface whereby a user entered the search query. In some implementations, the search query may be machine generated or received from another system.

At step 730, the mathematical model may be used to compute an embedding of the search query. In one implementation, the mathematical model may be a sentence embedding model, and the embedding of the search query may be a sentence embedding of the whole query. In one implementation, the embedding may be a transformer text embedding, and may be computed by a transformer neural network. In some implementations, the embedding may be computed from embeddings of portions of the query (e.g., a phrase, word, or token), such as by combining the embeddings of the portions (e.g., an average of the embeddings of the portions). At step 740, candidate embeddings in the embedding space that are in proximity to the embedding of the search query may be identified using a distance metric. The embedding space may be a combined embedding space and may include embeddings of language and molecule representations. Molecule representations may be text strings such as SMILES strings or SELFIES strings. Various distance metrics may be used, such as Euclidean distance, Manhattan distance, a cosine similarity metric, and the like.

A step 750, a candidate molecule, candidate molecule property, and/or text descriptions corresponding to at least one of the candidate embeddings may be obtained. In some cases, the candidate embedding may correspond to the closest embedding to the search query embedding based on the distance metric. In some cases, a plurality of candidate embeddings that correspond to the top number of closest candidate embeddings may be obtained.

At step 760, a representation of the candidate molecule(s), candidate molecule property, and/or text descriptions may be generated. The representation of the molecules, properties, and/or text descriptions may be displayed to a user via a user interface where the user may select and manipulate the results. The results may include data about the molecules retrieved from molecule databases or other sources. Embeddings corresponding to text descriptions of the candidate molecules may be identified and the corresponding text descriptions may be displayed with the representations of the candidate molecules.

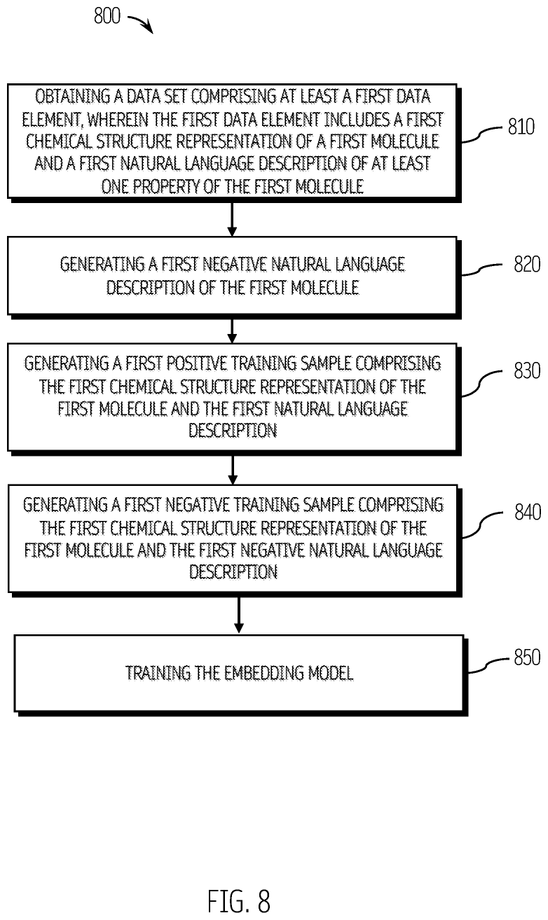

FIG. 8 is a flowchart of an example method 800 of training an embedding model. The trained embedding model may be a sentence embedding model. The trained embedding model may be a mathematical model for processing queries for molecules. In some implementations, training may include fine-tuning a pre-trained language model.

At step 810, a dataset may be obtained. The dataset may include one or more data elements and the data elements may include data such as a first chemical structure representation of a first molecule and a natural language description of at least one property of the first molecule.

In some implementations, training may include training on positive and negative samples. At step 820, a first negative natural language description of the first molecule may be generated. The negative natural language description may be generated or synthesized from the first natural language descriptions. At step 830, a first positive training sample may be generated. The first positive training sample may include the first chemical structure representation of the first molecule and the first natural language description. A step 840, a first negative training sample may be generated. The first negative training sample may include the first chemical structure representation of the first molecule and the first negative natural language description. The steps 820-840 of method 800 may be repeated for the plurality of data elements of the data set. A step 850, the model may be trained using the positive and/or negative training samples.

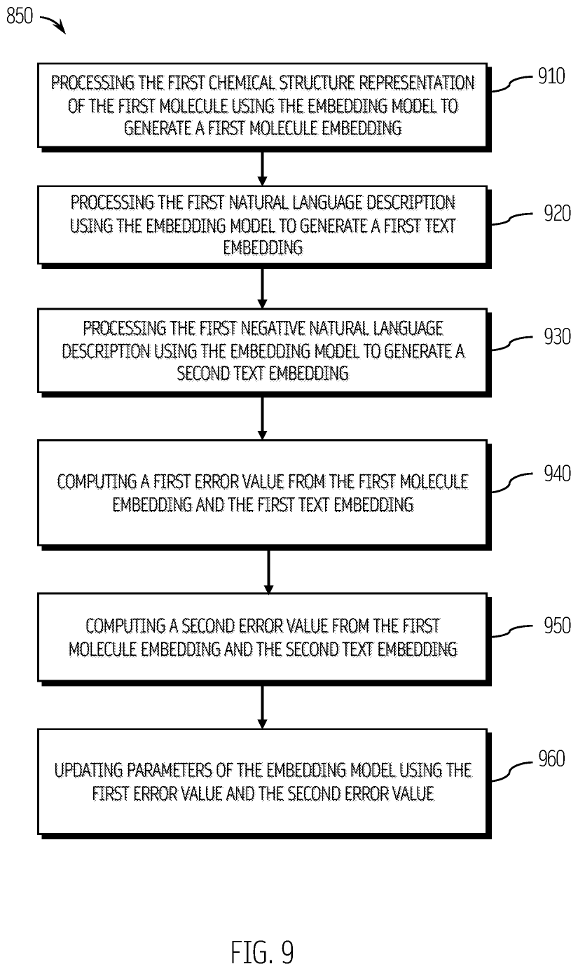

FIG. 9 is a flowchart showing details of an example training method of step 850 of FIG. 8. At step 910, the first chemical structure representation of the first molecule may be processed using the embedding model to generate a first molecule embedding. As step 920, the first natural language description may be processed using the embedding model to generate a first text embedding. At step 930, the first negative natural language description may be processed using the embedding model to generate a second text embedding.

At step 940, a first error value from the first molecule embedding and the first text embedding may be computed. At step 950, a second error value from the first molecule embedding and the second text embedding may be computed. The error values may be computed using a pseudo-normal negative loss function, triplet loss, or any other appropriate loss function. At step 960, the parameters of the embedding model may be updated using the first error value and second error value. The model parameters may be updated or fine-tuned to reduce the errors through stochastic gradient descent and backpropagation, or their alternatives. The training method of step 850 may be repeated for a plurality of positive and negative samples. In some implementations, the training data may be divided into subsets or ‘batches’, making it more computationally efficient than using the entire dataset at once. This iterative process, which may span one or more epochs, continues until the training reaches convergence—that is, the point at which the error ceases to decline or attains an acceptable threshold.

The methods and systems described herein provide a number of benefits in computer technology and in the fields of machine learning and automated chemical analysis.

In one aspect, validation of the methods described herein showed that the model may outperform previously known methods on datasets on which the model was trained on. In some implementations, the model that was trained with negative samples and the pseudo-normal negative loss function was shown to have high accuracy. The performance of the methods and systems described herein was shown to be, in many cases, more accurate than previously known methods based on message-passing neural network models (MPNN).

In another aspect, validation of the models has shown that the trained models described herein can generalize across tasks that the model has never been trained on and can learn aspects of the chemical space, which allow the models to make predictions about properties of molecules with high accuracy.

In another aspect, the use of combined embedding space allows the system to solve new kinds of problems that are not possible with classification models, like creating a search engine allowing a user to search for molecular properties using natural language.

In another aspect, the use of combined embedding space allows for simpler and faster system implementation requiring fewer trained models, which reduces training and maintenance requirements.

In yet another aspect, the methods and system described herein, allow improved user interfaces for searching and comparing molecules. The methods and systems described herein allow users to use natural language queries without requiring the user to be constrained by proprietary syntax or interfaces.

The methods and systems described herein may be deployed in part or in whole through a machine that executes computer software, program codes, and/or instructions on a processor. “Processor” as used herein is meant to include at least one processor and unless context clearly indicates otherwise, the plural and the singular should be understood to be interchangeable. Any aspects of the present disclosure may be implemented as a computer-implemented method on the machine, as a system or apparatus as part of or in relation to the machine, or as a computer program product embodied in a computer readable medium executing on one or more of the machines. The processor may be part of a server, server computer, client, network infrastructure, mobile computing platform, stationary computing platform, or other computing platform. A processor may be any kind of computational or processing device capable of executing program instructions, codes, binary instructions, and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more locations without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client, and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of program across the network. The networking of some or all of these devices may facilitate parallel processing of a program or method at one or more locations without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, circuits, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application-specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

All documents referenced herein are hereby incorporated by reference in their entirety.

1. A computer-implemented method of querying for molecules, comprising:
obtaining a mathematical model for embedding text and molecule representations in an embedding space;
receiving a natural language search query, wherein the search query includes a text description of a chemical property of a molecule;
computing, using the mathematical model, an embedding of the search query;
identifying, using a distance metric, a candidate embedding in the embedding space that is in proximity to the embedding of the search query;
obtaining a candidate molecule corresponding the candidate embedding; and
generating, for presentation at a user interface, a representation of the candidate molecule in response to the search query.

2. The method of claim 1, wherein computing the embedding of the search query comprises processing the text description with a sentence embedding model.

3. The method of claim 1, wherein the embedding is a transformer text embedding.

4. The method of claim 1, wherein the candidate embedding is representative of a SELFIES or a SMILES string of a molecule.

5. The method of claim 1, further comprising:
identifying, using the distance metric, a second candidate embedding in the embedding space that is in proximity to the embedding of the candidate molecule;
obtaining a property description corresponding to the second candidate embedding; and
generating, for presentation at the user interface, a depiction of the property description.

6. The method of claim 1, wherein the distance metric comprises cosine similarity.

7. The method of claim 1, wherein the representation of the candidate molecule comprises a graphical depiction of a structure of the molecule.

8. The method of claim 1, wherein the mathematical model comprises a transformer-based model.

9. The method of claim 1, wherein the mathematical model comprises a model trained using negative descriptions of molecules.

10. The method of claim 1, wherein the mathematical model comprises a model trained using a pseudo-normal negative loss function.

11. A computer-implemented method of querying for properties of a molecule, the method comprising:
receiving a molecule search query, wherein the molecule search query includes a text string representative of the molecule;
tokenizing the text string;
computing, using a mathematical model, an embedding of the tokenized string;
identifying, using a distance metric, a candidate embedding in an embedding space that is in proximity to the embedding of the tokenized string;
mapping the candidate embedding to text of a candidate molecule property; and
generating, for presentation at a user interface, a representation of the candidate molecule property.

12. The method of claim 11, wherein computing the embedding of the search query comprises processing the text string representation with a sentence embedding model.

13. The method of claim 11, wherein the embedding is a transformer text embedding.

14. The method of claim 11, wherein the candidate embedding is representative of a SELFIES or a SMILES string of a molecule.

15. The method of claim 11, wherein the distance metric comprises cosine similarity.

16. The method of claim 11, wherein the mathematical model comprises a model trained using negative descriptions of molecules.

17. A system, comprising:
at least one server computer comprising at least one processor and at least one memory, the at least one server computer configured to:
obtain a mathematical model for embedding text and molecule representations in an embedding space;
receive a natural language search query, wherein the search query includes a text description of a chemical property of a molecule;
compute, using the mathematical model, an embedding of the search query;
identify, using a distance metric, a candidate embedding in the embedding space that is in proximity to the embedding of the search query;
obtain a candidate molecule corresponding to the candidate embedding; and
generate, for presentation at a user interface, a representation of the candidate molecule in response to the search query.

18. The system of claim 17, wherein the at least one server computer is configured to compute the embedding of the text by processing the text with a sentence embedding model.

19. The system of claim 17, wherein the embedding space is a transformer embedding space.

20. The system of claim 17, wherein the candidate embedding is representative of a SELFIES or a SMILES string of a molecule.

21.-40. (canceled)
